(19) United States
(12) Patent Application Publication
(10) Pub. No.: US 2026/0151596 A1
(43) Pub. Date: Jun. 4, 2026

(54) HYBRID EXPANDABLE SHEATH

(71) Applicant: Edwards Lifesciences Corporation, Irvine, CA (US)

(72) Inventor: Richard D. White, Costa Mesa, CA (US)

(73) Assignee: Edwards Lifesciences Corporation, Irvine, CA (US)

(21) Appl. No.: 19/462,960

(22) Filed: Jan. 28, 2026

Related U.S. Application Data
(63) Continuation of application No. PCT/US2024/039895, filed on Jul. 26, 2024.
(60) Provisional application No. 63/530,924, filed on Aug. 4, 2023.

(51) Int. Cl. A61M 25/00

(52) U.S. Cl. CPC A61M 25/0045; A61M 25/005; A61M 25/0097; A61M 2025/0006; A61M 2025/0024

(57) ABSTRACT

The present disclosure is directed to a hybrid sheath system combining two sheaths using a connector region. The hybrid sheath system includes a proximal portion defining a first lumen and a distal portion defining a second lumen. A connector region couples the proximal portion to the distal portion to establish communication between the first and second lumens for passage of the medical device. Various implementations include a hybrid sheath system combining a non-expandable sheath and an expandable sheath with a connector region.

### CROSS-REFERENCE TO RELATED APPLICATIONS

This application is a continuation of International Application No. PCT/US2024/039895, filed Jul. 26, 2024, and entitled “HYBRID EXPANDABLE SHEATH,” claims the benefit of U.S. Provisional Application number 63/530,924, filed Aug. 4, 2023, the contents of which are incorporated by reference in their entirety.

### FIELD

The present application is directed to sheaths for delivery of medical devices and in particular expandable sheaths for delivery of medical devices.

### BACKGROUND

During a transcatheter procedure, a valve is crimped onto a delivery system, and the system is inserted into the patient vasculature via an access sheath and tracked to the target valve in the patient's heart. After deployment of the replacement valve, the delivery system is removed back through the access sheath. Use of an expandable access sheath mitigates vessel trauma and allows the physician to use a device that maintains a low profile before and after insertion of the delivery system, helping to reduce vascular complications. One tradeoff with expandable sheaths is that there is increased push force to insert the system through it versus a traditional sheath with a larger internal diameter.

Accordingly, there remains a need for devices systems, and methods providing delivery sheaths for implants.

### SUMMARY

Implementations of the present expandable sheath system can minimize trauma to the vessel and damage to the sheath and prosthetic device by reducing push forces through the blood vessel and/or sheath.

An example sheath system according to the present disclosure includes a hybrid sheath system including a combination of two sheaths using a connector region.

In some examples, the hybrid sheath may be for deploying a medical device and include a proximal portion defining a first lumen and a distal expandable portion defining a second lumen.

In some examples, the proximal portion includes at least one polymer layer defining the first lumen with a fixed diameter.

In some examples, the distal portion includes an expandable layer combined with a first polymeric layer.

In some examples, the connector region can be configured to couple a proximal end of the expandable distal portion to a distal end of the proximal portion to establish communication between the first and second lumens for passage of the medical device.

In some examples, the expandable distal portion further includes: an inner liner including at least one polymeric layer having an inner surface defining the second lumen; at least one second polymeric layer surrounding the inner liner and wherein the expandable layer surrounds the second polymeric layer; and an outer liner including at least one polymeric layer surrounding the first polymeric layer. In some examples, the first polymeric layer surrounds the expandable layer, and the inner liner, the second polymeric layer, the expandable layer, the first polymeric layer and the outer liner form a laminate structure configured to expand for passage of the medical device through the second lumen.

In some examples, the hybrid sheath further includes a hub coupled to a proximal end of the proximal portion, the hub configured to engage a stabilizer to resist push forces through the first and second lumens.

In some examples, the connector region includes an extension of one of the polymeric layers of the expandable distal portion over the distal end of the proximal portion.

In some examples, the extension of one of the polymeric layers is bonded to the distal end of the proximal portion.

In some examples, the one of the polymeric layers is the first polymeric layer.

In some examples, the distal end of the proximal portion extends between the first polymeric layer and the expandable layer at the connector region.

In some examples, an inner surface of the distal end of the proximal portion is bonded directly to the expandable layer and wherein an inner surface of the first polymeric layer is bonded directly to the distal end of the proximal portion at the connector region.

In some examples, the first polymeric layer is reflowed onto the distal end of the proximal portion with the expandable layer between the first polymeric layer and the distal end of the proximal portion.

In some examples, the polymer layer of the proximal portion is a rigid layer.

In some examples, the expandable layer includes a flexible mesh layer.

In some examples, the flexible mesh layer includes a braided layer.

In some examples, the expandable layer includes a folded layer.

In some examples, the proximal portion includes inner and outer polymer layers, where the inner polymer layer of the proximal portion defines the first lumen and where the expandable layer extends between the inner and outer polymer layers of the proximal portion at the connector region.

In some examples, the first lumen of the proximal portion is 28 French or greater.

In some examples, a length of the proximal portion is at least 17 inches.

In some examples, the second lumen of the expandable distal portion has a non-expanded diameter of 20 French or less.

Various aspects of the implementations described above can be combined based on desired sheath system characteristics.

### BRIEF DESCRIPTION OF DRAWINGS

FIG. 1 is an elevation view of a hybrid sheath along with an endovascular delivery apparatus for implanting a prosthetic implant.

FIG. 2 is a side elevation view of the hybrid sheath of FIG. 1 including a proximal portion and a distal expandable portion.

FIG. 3 is cross-sectional view of a connector region of the hybrid sheath of FIG. 2.

FIG. 4 is a side elevation cross-sectional view of a distal expandable portion of the hybrid sheath of FIGS. 1 and 2.

FIG. 5 is a magnified view of a distal expandable portion of the hybrid sheath of FIGS. 1 and 2.

FIG. 6A is a magnified view of a distal expandable portion of the hybrid sheath of FIGS. 1 and 2 with the outer layer removed for purposes of illustration.

FIG. 6B is a magnified view of a distal expandable portion of the braided layer of the distal expandable portion of the hybrid sheath of FIGS. 1 and 2.

FIG. 7 is a magnified view of the expandable portion of the hybrid sheath of FIGS. 1 and 2 illustrating expansion of the sheath as a prosthetic device is advanced through the sheath.

FIGS. 8-13 are cross-sectional schematics of various implementations of connector regions of hybrid sheaths.

### DETAILED DESCRIPTION

The following description of certain examples of the inventive concepts should not be used to limit the scope of the claims. Other examples, features, aspects, implementations, and advantages will become apparent to those skilled in the art from the following description. As will be realized, the device and/or methods are capable of other different and obvious aspects, all without departing from the spirit of the inventive concepts. Accordingly, the drawings and descriptions should be regarded as illustrative in nature and not restrictive.

For purposes of this description, certain aspects, advantages, and novel features of the aspects of this disclosure are described herein. The described methods, systems, and apparatus should not be construed as limiting in any way. Instead, the present disclosure is directed toward all novel and nonobvious features and aspects of the various disclosed aspects, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Features, integers, characteristics, compounds, chemical moieties, or groups described in conjunction with a particular aspect or example of the present disclosure are to be understood to be applicable to any other aspect or example described herein unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The present disclosure is not restricted to the details of any foregoing aspects. The present disclosure extends to any novel one, or any novel combination, of the features disclosed in this specification (including any accompanying claims, abstract, and drawings), or to any novel one, or any novel combination, of the steps of any method or process so disclosed.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

The terms “proximal” and “distal” as used herein refer to regions of a sheath, catheter, or delivery assembly. “Proximal” means that region closest to handle of the device, while “distal” means that region farthest away from the handle of the device.

“Axially” or “axial” as used herein refers to a direction along the longitudinal axis of the sheath.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

Disclosed aspects of a hybrid sheath with an expandable distal portion for insertion into a patient body that can minimize trauma to the vessel by allowing for temporary expansion of a portion of the introducer sheath to accommodate the delivery system, followed by a return to the original diameter once the device passes through. The hybrid sheath also includes a non-expandable proximal portion that dwells outside the patient. The non-expandable proximal portion mediates the amount of push force needed to advance the medical device (such as an expandable heart valve in a capsule) through the patient. In addition, the non-expandable proximal portion can include a proximal hub that can be held in place by a stabilizer. In one use or advantage, the sheath would be inserted into the patient, the delivery system pushed through the sheath, and then the sheath retracted and docked into the stabilizer, such that the expandable region remains in the patient and the (generally more) rigid portion is outside of the patient. In another use or advantage, the stabilizer prevents movement during the procedure, especially when the push forces climb during expansion of the expandable distal portion of the hybrid sheath.

Disclosed herein are elongated introducer sheaths that are particularly suitable for delivery of implants in the form of implantable heart valves, such as balloon-expandable implantable heart valves. Balloon-expandable implantable heart valves are well-known and will not be described in detail here. An example of such an implantable heart valve is described in U.S. Pat. No. 5,411,552, and also in U.S. Pat. No. 9,393,110, both of which are hereby incorporated by reference. The expandable introducer sheaths disclosed herein may also be used to deliver other types of implantable medical device, such as self-expanding and mechanically expanding implantable heart valves, stents or filters. Beyond transcatheter heart valves, the introducer sheath system can be useful for other types of minimally invasive surgery, such as any surgery requiring introduction of an apparatus into a subject's vessel. For example, the introducer sheath system can be used to introduce other types of delivery apparatus for placing various types of intraluminal devices (for example, stents, stented grafts, balloon catheters for angioplasty procedures, etc.) into many types of vascular and non-vascular body lumens (for example, veins, arteries, esophagus, ducts of the biliary tree, intestine, urethra, fallopian tube, other endocrine or exocrine ducts, etc.). The term “implantable” as used herein is broadly defined to mean anything - prosthetic or not - that is delivered to a site within a body. A diagnostic device, for example, may be an implantable device.

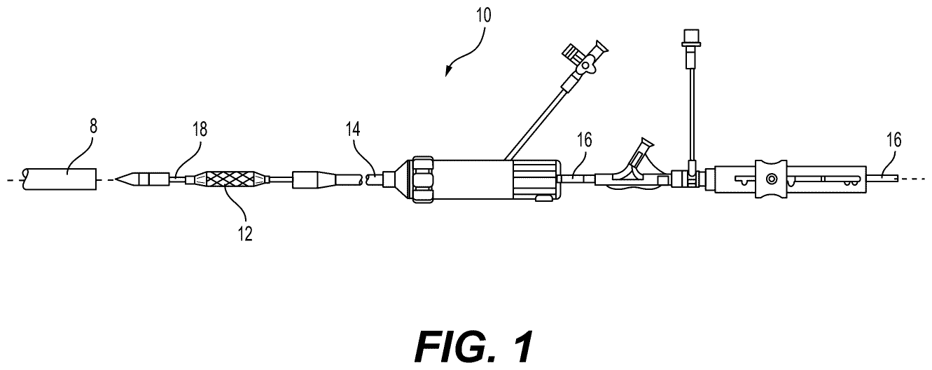

FIG. 1 illustrates an exemplary hybrid sheath 8 in use with a representative delivery apparatus 10, for delivering an implant 12, or other type of implantable (for example, tissue heart valve), to a patient. The delivery apparatus 10 can include a steerable guide catheter 14 (also referred to as a flex catheter) and a balloon catheter 16 extending through the guide catheter 14, and a nose catheter 18 extending through the balloon catheter 16. The guide catheter 14, balloon catheter 16, and nose catheter 18 in the illustrated example are adapted to slide longitudinally relative to each other to facilitate delivery and positioning of the implant 12 at an implantation site in a patient's body as described in detail herein. It is contemplated that the sheath 8 can be used with any type of elongated delivery apparatus used for implanting balloon-expandable prosthetic valves, self-expanding prosthetic valves, and other prosthetic devices.

As described in more detail herein, in general, the hybrid sheath 8 comprises an elongate hybrid (proximally rigid and distally expandable) tube that, in use, is inserted into a vessel (for example, transfemoral vessel, femoral artery, iliac artery) by passing through the skin of patient, such that the distal end of the hybrid sheath 8 is inserted into the vessel. Hybrid sheath 8 optionally includes a hemostasis valve and/or sealing features at the proximal end of the hybrid sheath 8, for example, in a sheath hub (for example, hub 20), that provide hemostasis and prevents blood leakage from the patient through the hybrid sheath 8. The hybrid sheath 8, including an introducer, is advanced into the patient's vasculature. Once positioned the introducer is removed and the delivery apparatus 10 is inserted into/through the hybrid sheath 8, and the prosthetic device (implant 12) then be delivered and implanted within patient.

As described herein, the introducer device/sheath assembly includes the hybrid sheath 8 extending distally from the sheath hub (for example, hub 20). The hybrid sheath 8 has a central lumen to guide passage of the delivery apparatus 10 for the medical device (implant 12/prosthetic heart valve). In some implementations, the introducer device/sheath assembly need not include a sheath hub. For example, the hybrid sheath 8 can be an integral part and/or component of the sheath assembly, such as the guide catheter. As described herein, the distal expandable portion of the hybrid sheath 8 can have a natural, unexpanded outer diameter that will expand locally upon passage of the medical device (implant 12/prosthetic heart valve).

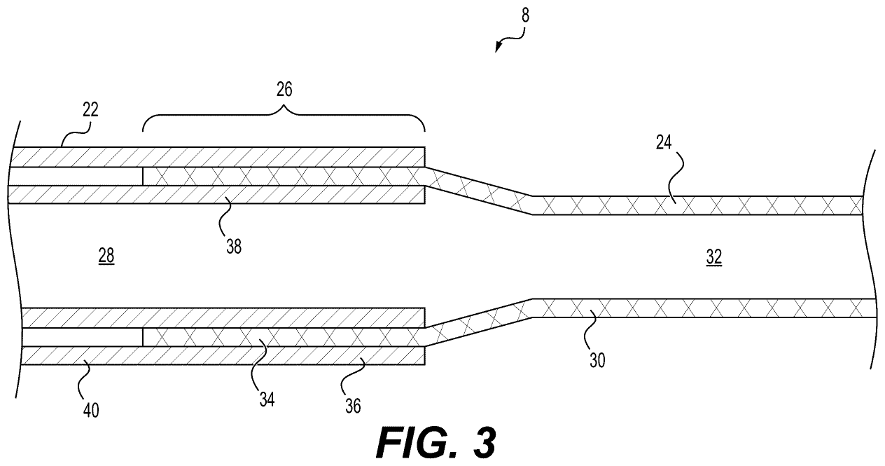

In some implementations, the hybrid sheath 8 can comprise a plurality of coaxial layers extending along at least a portion of the length of the hybrid sheath 8, especially the distal expandable portion. The structure of the coaxial layers is described in more detail herein with respect to FIGS. 3-13. Example expandable sheaths including coaxial layers are described, for example, in U.S. patent application Ser. No. 16/378,417, entitled “Expandable Sheath,” and U.S. patent application Ser. No. 17/716,882, entitled “Expandable Sheath,” the disclosures of which are herein incorporated by reference.

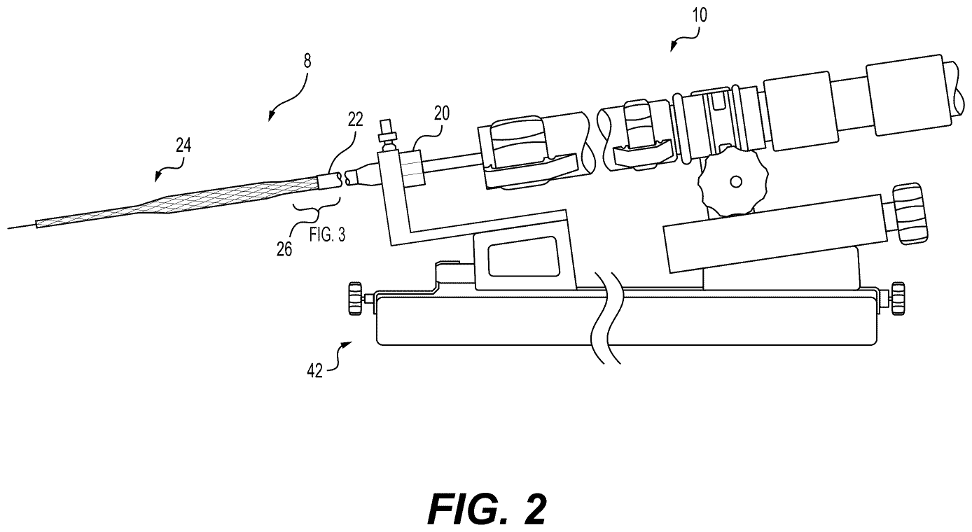

As shown in FIG. 2, the hybrid sheath 8 includes a proximal portion 22 and an expandable distal portion 24 that are connected by a connector region 26. The proximal portion includes one or more polymer layers defining a first lumen 28 having a generally fixed diameter to reduce push forces for the portion of the hybrid sheath 8 outside of the patient. As described further herein with respect to FIG. 3, the distal portion 24 includes one or more expandable layers 30 and inner polymeric and outer polymeric layers that overall define a second lumen 32.

FIG. 3 shows an enlarged cross-sectional view of the hybrid sheath 8. As shown, the connector region 26 includes a proximal end 34 of the expandable distal portion 24 and a distal end 36 of the non-expandable proximal portion 22, as shown in FIG. 3. The connector region 26 is configured to couple the proximal end 34 of the expandable distal portion 24 to the distal end 36 of the proximal portion 22 to establish communication between the first and second lumens 28 and 32. During use, the implant 12 can be advanced through the combined lumen, first through the proximal portion 22 with its relatively large diameter and then through the expandable distal portion 24, mostly within the patient. In this manner the connector region 26 can combine the advantages of a fixed-diameter introducer region outside the patient with an expandable introducer region, and its initially small profile, inside the patient.

The expandable distal portion 24 can include one of several structures for enabling expansion for passage of the implant 12. For example, the expandable distal portion can include the expandable layer 30 in the form of a mesh or braided layer or in the form of a foldable layer or a split layer, examples of which are described in more detail herein.

The proximal portion 22 generally is not functionally expandable in that it responds little to passage of the medical device or other implant 12 in contrast to the expandable layer 30 which can expand substantially, and even contract, in response to passage of the implant 12. Thus, the term “fixed diameter” as used herein is referring to a diameter that does not change substantially in response to passage of an implant or other device and that is sized to provide less resistance to push through of the implant. For example, the proximal portion may be a somewhat rigid dual-layered polymeric tube with an inner layer 38 and an outer layer 40. As shown in FIG. 3, the outer layer 40 defines an outer surface and the inner layer 38 defines the first lumen 28. Together these layers are stiff enough to provide clearance for easier passage of the implant 12.

In some implementations, the proximal portion 22 can include a fixed inner diameter with an optional PTFE layer for friction reduction. This could be a continuous liner through both the proximal and distal portions 22, 24. The outer layer 40 can be a fixed diameter, optionally constructed from a PEBAX polymer. Between these layers can be a fixed diameter braid or coil middle layer, for example a stainless-steel braid or coil. The proximal portion 22 (or shaft), along with the distal portion 24, can include an optional hydrophilic coating. For example, a single dipping process can coat the entire hybrid sheath 8.

The connector region 26 can include the overlapping portions of the proximal portion 22 and the distal portion 24 of the hybrid sheath 8. For example, in FIG. 3, the overlapping portions include the proximal end 34 of the distal portion 24 and the distal end 36 of the proximal portion 22. These portions are coupled to each other, such as by use of polymeric properties - such as by heat reflowing the polymeric layers into a laminate structure. For example, the expandable distal portion is expanded at its proximal end 34 to sleeve over the distal end 36 of the inner layer 38 of the non-expandable proximal portion 22. In this configuration, the proximal end 34 of the expandable distal portion 24 is “sandwiched” or layered in between the inner layer 38 and outer layer 40 of the proximal portion 22. These layers can then be laminated to each other by adhesive or flow melting of the polymeric layers. Heat shrink tubing may also be sleeved over the overlapping portions of the connector region 26. In some implementations, the layers may also and/or alternatively be mechanically fastened to each other, such as with sutures or staples.

The connector region 26 may also be formed with other configurations, such as by interleaving various layers of the proximal end 34 and distal ends 36 and forming a laminate structure. Layers also need not be coextensive with each other, for example, the inner layer 38 and outer layer 40 can have staggered terminations for the distal end 36 of the proximal portion 22 so that the inner layer 38 projects further into the proximal end 34 of the distal portion 24. Choosing which layers extend which distance and how the layers overlap and interleave and how the layers are attached to each other allow for modulation of strength of the connection, the stiffness profile along the axis of the hybrid sheath 8 and the tradeoffs between push resistance and expandability. In addition, folding layers can be expanded progressively into the connector region 26 to adjust for the transition to the expandable, second lumen 32.

As shown in FIG. 2, the hybrid sheath 8 can also include a hub 20. The hub 20 is attached to a proximal end of the proximal portion 22. The hub 20, as well as containing a valve and having other structure and function described herein, can be coupled to a stabilizer 42. Advantageously, the hybrid sheath 8 could be inserted into the patient, the delivery system pushed through the hybrid sheath 8, and then the hybrid sheath 8 retracted and docked into the stabilizer, such that the expandable distal portion 24 remains in the patient and the proximal portion 22 is outside of the patient. Engagement of the stabilizer can also help to mediate or resist push forces from advancing the implant 12 through the first lumen 28 and second lumen 32.

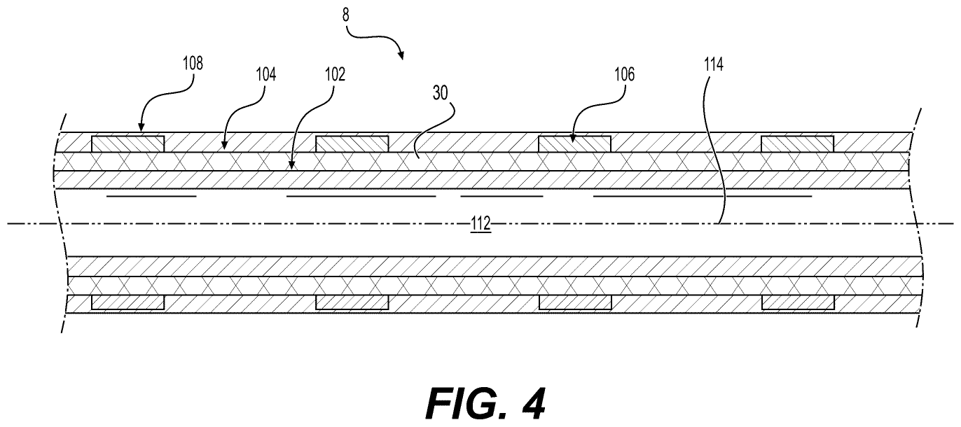

Various implementations of the coaxial layered structure of the expandable distal portion 24 are described herein. For example, in reference to the example distal portion 24 illustrated in FIGS. 4-7, the expandable distal portion 24 can include a number of layers including an inner layer 102 (also referred to as an inner layer), a second layer 104 disposed around and radially outward of the inner layer 102, a third layer 106 disposed around and radially outward of the second layer 104, and a fourth outer layer 108 (also referred to as an outer layer) disposed around and radially outward of the third layer 106. In the illustrated configuration, the inner layer 102 can define the lumen 112 of the hybrid sheath 8 extending along a central axis 114 through which the delivery apparatus (for example, delivery apparatus 10) travels into the patient's vessel in order to deliver, remove, repair, and/or replace a prosthetic device (implant 12), moving in a direction along the longitudinal axis of the distal portion 24.

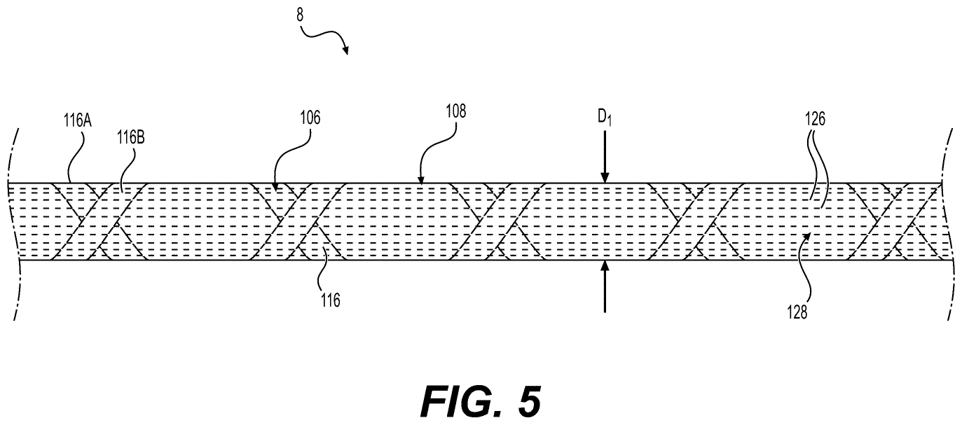

Referring to FIG. 5, when the distal portion 24 is in an unexpanded state, various layers of the hybrid sheath 8, for example, the inner layer 102 and/or the outer layer 108, can form longitudinally-extending folds or creases such that the surface of the hybrid sheath 8 comprises a plurality of ridges 126 (also referred to herein as “folds”). The ridges 126 can be circumferentially spaced apart from each other by longitudinally-extending valleys 128. When the hybrid sheath 8 expands beyond its natural diameter D1, the ridges 126 and the valleys 128 can level out or be taken up as the surface radially expands and the circumference increases, as further described herein. When the distal portion 24 collapses back to its natural diameter, the ridges 126 and valleys 128 can reform.

In some implementations, the inner layer 102 and/or the outer layer 108 can comprise a relatively thin layer of polymeric material. For example, in some implementations, the thickness of the inner layer 102 can be from 0.01 mm to 0.5 mm, 0.02 mm to 0.4 mm, or 0.03 mm to 0.25 mm. In some implementations, the thickness of the outer layer 108 can be from 0.01 mm to 0.5 mm, 0.02 mm to 0.4 mm, or 0.03 mm to 0.25 mm.

In some implementations, the inner layer 102 and/or the outer layer 108 can optionally comprise a lubricious, low-friction, and/or relatively non-elastic material. In some implementations, the inner layer 102 and/or the outer layer 108 can comprise a polymeric material having a modulus of elasticity of 400 MPa or greater. Exemplary materials can include ultra-high-molecular-weight polyethylene (UHMWPE) (for example, Dyneema®), high-molecular-weight polyethylene (HMWPE), or polyether ether ketone (PEEK). With regard to the inner layer 102 in particular, such low coefficient of friction materials can facilitate passage of the prosthetic device (implant 12) through the lumen 112. Other suitable materials for the inner layer 102 and/or outer layer 108 can include polyimide, polytetrafluoroethylene (PTFE), expanded polytetrafluoroethylene (ePTFE), ethylene tetrafluoroethylene (ETFE), nylon, polyethylene, polyamide, polyether block amide (for example, Pebax), and/or combinations of any of the herein. Some implementations of the distal portion 24 can optionally include a lubricious liner on the inner surface of the inner layer 102. Examples of suitable lubricious liners include materials that can further reduce the coefficient of friction of the inner layer 102, such as PTFE, polyethylene, polyvinylidene fluoride, and combinations thereof. Suitable materials for a lubricious liner also include other materials desirably having a coefficient of friction of 0.1 or less.

Additionally, some implementations of the distal portion 24 can optionally include an exterior hydrophilic coating on the outer surface of the outer layer 108. Such a hydrophilic coating can facilitate insertion of the distal portion 24 into a patient's vessel, reducing potential damage. Examples of suitable hydrophilic coatings include the Harmony™ Advanced Lubricity Coatings and other Advanced Hydrophilic Coatings available from SurModics, Inc., Eden Prairie, MN. DSM medical coatings (available from Koninklijke DSM NV, Heerlen, the Netherlands), as well as other hydrophilic coatings (for example, PTFE, polyethylene, polyvinylidene fluoride), are also suitable for use with the distal portion 24. Such hydrophilic coatings may also be included on the inner surface of the inner layer 102 to reduce friction between the hybrid sheath 8 and the delivery system, thereby facilitating use and improving safety. In some implementations, an optional hydrophobic coating, such as Perylene, may be used on the outer surface of the outer layer 108 or the inner surface of the inner layer 102 in order to reduce friction.

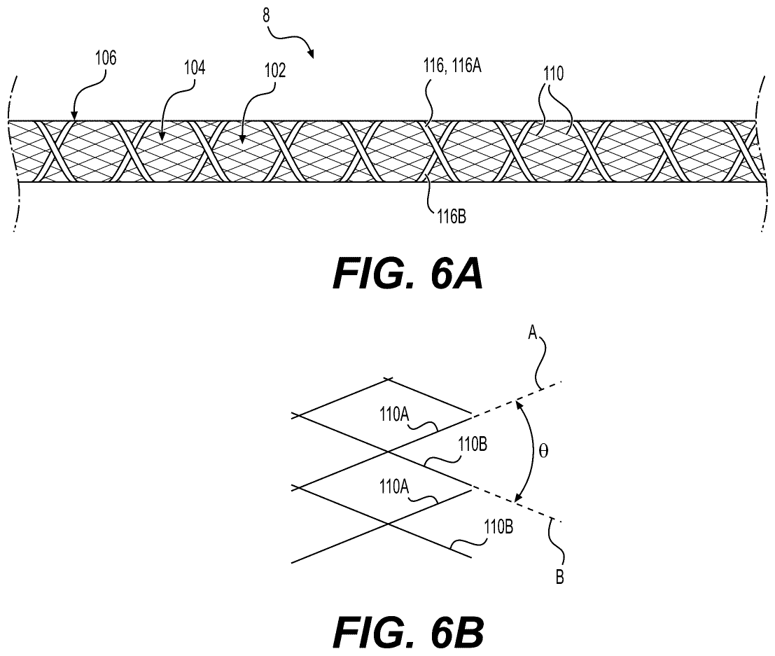

In some implementations, the second layer 104 can be a braided layer. FIGS. 6A and 6B illustrate the distal portion 24 with the outer layer 108 removed to expose the elastic third layer 106. With reference to FIGS. 6A and 6B, the braided second layer 104 can comprise a plurality of members or filaments 110 (for example, metallic or synthetic wires or fibers) braided together. The braided second layer 104 can have any desired number of filaments 110, which can be oriented and braided together along any suitable number of axes. For example, with reference to FIG. 6B, the filaments 110 can include a first set of filaments 110A oriented parallel to a first axis A, and a second set of filaments 110B oriented parallel to a second axis B.

The filaments 110A and 110B can be braided together in a biaxial braid such that filaments 110A oriented along axis A form an angle θ with the filaments 110B oriented along axis B. In some implementations, the angle θ can be from 5° to 70°, 10° to 60°, 10° to 50°, or 10° to 45°. In the illustrated example, the angle θ is 45°. In some implementations, the filaments 110 can also be oriented along three axes and braided in a triaxial braid, or oriented along any number of axes and braided in any suitable braid pattern. The braided second layer 104 can extend along substantially the entire length L of the distal portion 24, or alternatively, can extend only along a portion of the length of the hybrid sheath 8. In some implementations, the filaments 110 can be wires made from metal (for example, Nitinol, stainless steel, etc.), or any of various polymers or polymer composite materials, such as carbon fiber.

In some implementations, the filaments 110 can be round, and can have a diameter of from 0.01 mm to 0.5 mm, 0.03 mm to 0.4 mm, or 0.05 mm to 0.25 mm. In some implementations, the filaments 110 can have a flat cross-section with dimensions of 0.01 mm×0.01 mm to 0.5 mm×0.5 mm, or 0.05 mm×0.05 mm to 0.25 mm×0.25 mm. In some implementations, filaments 110 having a flat cross-section can have dimensions of 0.1 mm×0.2 mm. However, other geometries and sizes are also suitable for some implementations. If braided wire is used, the braid density can be varied. Some implementations have a braid density of from ten picks per inch to eighty picks per inch, and can include eight wires, sixteen wires, or up to fifty-two wires in various braid patterns. In some implementations, the second layer 104 can be laser cut from a tube, or laser-cut, stamped, punched, etc., from sheet stock and rolled into a tubular configuration. The second layer 104 can also be woven or knitted, as desired.

The third layer 106 can be a resilient, elastic layer (also referred to as an elastic material layer). In some implementations, the elastic third layer 106 can be configured to apply radially inward force to the underlying inner layer 102 and second layer 104 in a radial direction (for example, toward the central axis 114 of the hybrid sheath 8) when the hybrid sheath 8 expands beyond its natural diameter by passage of the delivery apparatus (for example, delivery apparatus 10) through the hybrid sheath 8. Stated differently, the elastic third layer 106 can be configured to apply encircling/radially inward pressure to the layers of the hybrid sheath 8 beneath the elastic third layer 106 to counteract expansion of the hybrid sheath 8. The radially inwardly directed force is sufficient to cause the hybrid sheath 8 to collapse radially back to its unexpanded state after the delivery apparatus (for example, delivery apparatus 10) is passed through the hybrid sheath 8.

In the illustrated example, the elastic third layer 106 can comprise one or more members configured as strands, ribbons, or bands 116 helically wrapped around the braided second layer 104. For example, in the illustrated implementation, the elastic third layer 106 comprises two elastic bands 116A and 116B wrapped around the braided second layer 104 with opposite helicity, although the elastic layer may comprise any number of bands depending upon the desired characteristics. The elastic bands 116A and 116B can be made from, for example, any of a variety of natural or synthetic elastomers, including silicone rubber, natural rubber, any of various thermoplastic elastomers, polyurethanes such as polyurethane siloxane copolymers, urethane, plasticized polyvinyl chloride (PVC), styrenic block copolymers, polyolefin elastomers, etc. In some implementations, the elastic layer can comprise an elastomeric material having a modulus of elasticity of 200 MPa or less. In some implementations, the elastic third layer 106 can comprise a material exhibiting an elongation to break of 200% or greater, or an elongation to break of 400% or greater. The elastic third layer 106 can also take other forms, such as a tubular layer comprising an elastomeric material, a mesh, a shrinkable polymer layer such as a heat-shrink tubing layer, etc.

In lieu of, or in addition to, the elastic third layer 106, the distal portion 24 may also include an elastomeric or heat-shrink tubing layer around the outer layer 108. Examples of such elastomeric layers are disclosed in U.S. Publication No. 2014/0379067, U.S. Publication No. 2016/0296730, and U.S. Publication No. 2018/0008407, which are incorporated herein by reference. In some implementations, the elastic third layer 106 can also be radially outward of the polymeric outer layer 108.

In some implementations, one or both of the inner layer 102 and/or the outer layer 108 can be configured to resist axial elongation of the distal portion 24 when the hybrid sheath 8 expands. More particularly, one or both of the inner layer 102 and/or the outer layer 108 can resist stretching against longitudinal forces caused by friction between a prosthetic device (implant 12) and the inner surface of the distal portion 24 such that the length L remains substantially constant as the hybrid sheath 8 expands and contracts. As used herein with reference to the length L of the hybrid sheath 8, the term “substantially constant” means that the length L of the hybrid sheath 8 increases by not more than 1%, by not more than 5%, by not more than 10%, by not more than 15%, or by not more than 20%. Meanwhile, with reference to FIG. 6B, the filaments 110A and 110B of the braided second layer 104 can be allowed to move angularly relative to each other such that the angle θ changes as the hybrid sheath 8 expands and contracts. This, in combination with the longitudinal ridges 126 (folds) in the inner layer 102 and outer layer 108, can allow the lumen 112 of the hybrid sheath 8 to expand as a prosthetic device (implant 12) is advanced through it.

For example, in some implementations, the inner layer 102 and the outer layer 108 can be heat-bonded during the manufacturing process such that the braided second layer 104 and the elastic third layer 106 are encapsulated between the inner layer 102 and outer layer 108. More specifically, in some implementations, the inner layer 102 and the outer layer 108 can be adhered to each other through the spaces between the filaments 110 of the braided second layer 104 and/or the spaces between the elastic bands 116. The inner layer 102 and outer layer 108 can also be bonded or adhered together at the proximal end and/or distal end of the hybrid sheath 8. In some implementations, the inner layer 102 and outer layer 108 are not adhered to the filaments 110. This can allow the filaments 110 to move angularly relative to each other, and relative to the inner layer 102 and outer layer 108, allowing the diameter of the braided second layer 104, and thereby the diameter of the hybrid sheath 8, to increase or decrease. As the angle θ between the filaments 110A and 110B changes, the length of the braided second layer 104 can also change. For example, as the angle θ increases, the braided second layer 104 can foreshorten, and as the angle θ decreases, the braided second layer 104 can lengthen to the extent permitted by the areas where the inner layer 102 and outer layer 108 are bonded. However, because the braided second layer 104 is not adhered to the inner layer 102 and outer layer 108, the change in length of the braided layer that accompanies a change in the angle θ between the filaments 110A and 110B does not result in a significant change in the length L of the hybrid sheath 8.

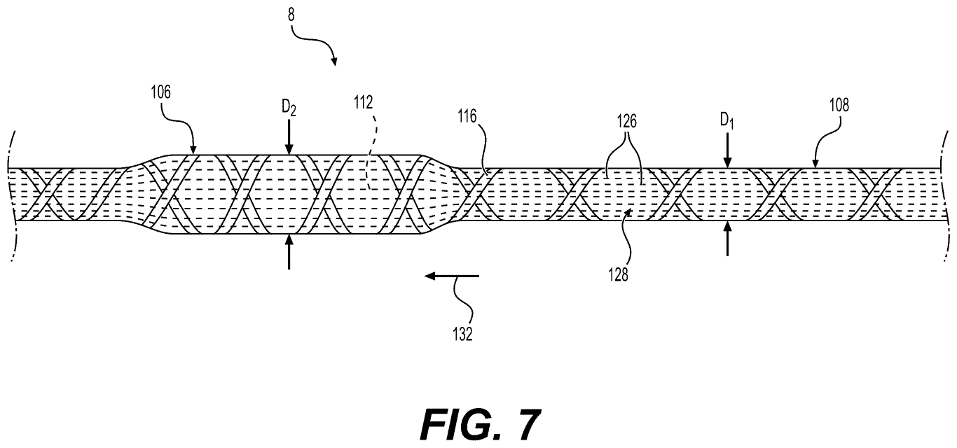

FIG. 7 illustrates radial expansion of the distal portion 24 as a prosthetic device (for example, implant 12) is passed through the distal portion 24 in the direction of arrow 132 (for example, distally). As the prosthetic device (implant 12) is advanced through the distal portion 24, the hybrid sheath 8 can resiliently expand to a second diameter D2 that corresponds to a size or diameter of the prosthetic device (implant 12). As the prosthetic device (implant 12) is advanced through the distal portion 24, the prosthetic device (implant 12) can apply longitudinal force to the hybrid sheath 8 in the direction of motion by virtue of the frictional contact between the prosthetic device (implant 12) and the inner surface of the hybrid sheath 8. However, as noted herein, the inner layer 102 and/or the outer layer 108 can resist axial elongation such that the length L of the hybrid sheath 8 remains constant, or substantially constant. This can reduce or prevent the braided second layer 104 from lengthening, and thereby constricting the lumen 112.

Meanwhile, the angle θ between the filaments 110A and 110B can increase as the hybrid sheath 8 expands to the second diameter D2 to accommodate the prosthetic valve (implant 12). This can cause the braided second layer 104 to foreshorten. However, because the filaments 110 are not engaged or adhered to the inner layer 102 or outer layer 108, the shortening of the braided second layer 104 attendant to an increase in the angle θ does not affect the overall length L of the hybrid sheath 8. Moreover, because of the longitudinally-extending ridges 126 (folds) formed in the inner layer 102 and outer layer 108, the inner layer 102 and outer layer 108 can expand to the second diameter D2 without rupturing, in spite of being relatively thin and relatively non-elastic. In this manner, the distal portion 24 can resiliently expand from its natural diameter D1 to a second diameter D2 that is larger than the diameter D1 as a prosthetic device (implant 12) is advanced through the hybrid sheath 8, without lengthening, and without constricting. Thus, the force required to push the prosthetic device (implant 12) through the hybrid sheath 8 is significantly reduced.

Additionally, because of the radial force applied by the elastic third layer 106, the radial expansion of the distal portion 24 can be localized to the specific portion of the hybrid sheath 8 occupied by the prosthetic device (implant 12). For example, with reference to FIG. 7, as the prosthetic device (implant 12) moves distally through the distal portion 24, the portion of the hybrid sheath 8 immediately proximal to the prosthetic device (for example, implant 12) can radially collapse back to the initial diameter D1 under the influence of the elastic third layer 106. The inner layer 102 and outer layer 108 can also buckle as the circumference of the hybrid sheath 8 is reduced, causing the ridges 126 and the valleys 128 to reform. This can reduce the size of the hybrid sheath 8 required to introduce a prosthetic device (implant 12) of a given size. Additionally, the temporary, localized nature of the expansion can reduce trauma to the blood vessel into which the hybrid sheath 8 is inserted, along with the surrounding tissue, because only the portion of the hybrid sheath 8 occupied by the prosthetic device (implant 12) expands beyond the hybrid sheath's 8 natural diameter and the hybrid sheath 8 collapses back to the initial diameter once the device (implant 12) has passed. This limits the amount of tissue that must be stretched in order to introduce the prosthetic device (implant 12), and the amount of time for which a given portion of the vessel must be dilated.

As noted, there are many ways to join the flexible/expandable sheath distal portion 24 to the proximal portion 22 with the more stable internal diameter to form a hybrid sheath 8. For example, as provided herein, the expandable layer 30 can include a braided layer or braided region. In some examples, the proximal end 34 of a braided region of the expandable sheath distal portion 24 (for example, expandable layer 30) is overlapped with a more rigid proximal sheath (for example, proximal portion 22). The outer layer 40 of the rigid sheath (proximal portion 22) is floated over the braided region (for example expandable layer 30). Fluorinated Ethylene Propylene heat shrink tubing can be shrunk into place over the joint and heat applied to reflow the outer layer 40 of rigid sheath (proximal portion 22) into the braid of the expandable sheath distal portion 24 (for example, expandable layer 30).

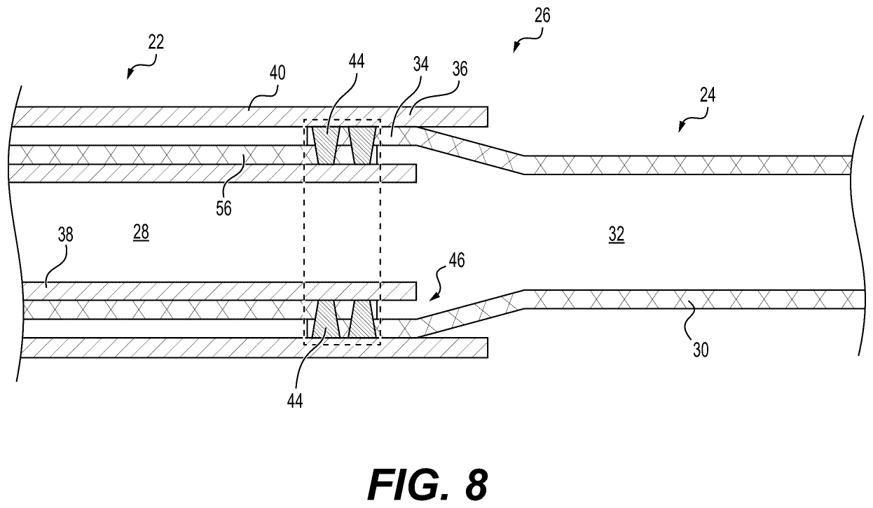

In some implementations, as shown in FIG. 8, a locking ring of rivets may be applied around proximal open cells of an expandable sheath (for example, expandable layer 30 of the distal portion 24). The ring of rivets 44 is pressed into a bottom ring 46 of a rigid proximal portion 22. The rivets can interlock between the braid cells of a reinforcing metal braid 56. The rings can be locked into place by welding or by press fitting. The outer layer 40 of the proximal portion 22 can be reflowed over the joint to cover the exposed area and make it atraumatic.

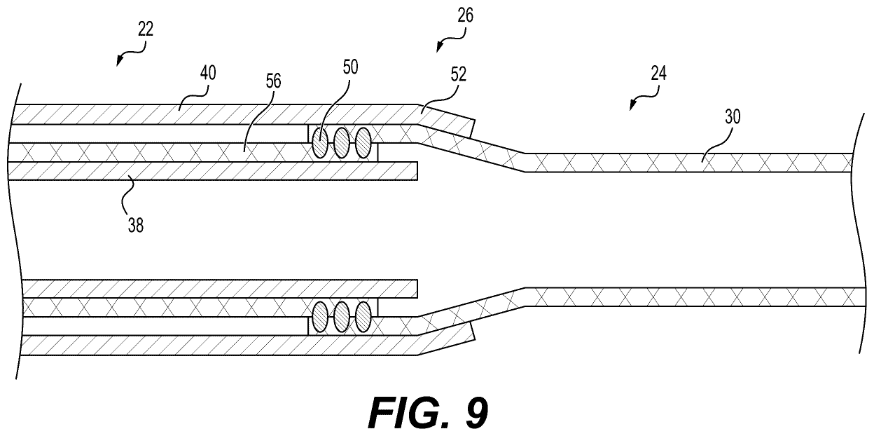

In some implementations, as shown in FIG. 9, a braided expandable layer 30 may be comprised of NiTi and is welded to the proximal portion 22 which comprises a rigid polymeric inner layer 38. In some implementations, the braided expandable layer 30 may be comprised of stainless steel or other biocompatible metallic materials that can be welded. The welds include single spot sized tack welds 50 to fix the braided expandable layer 30 into place. An additional collar 52 can be reflowed to keep the tacks in place.

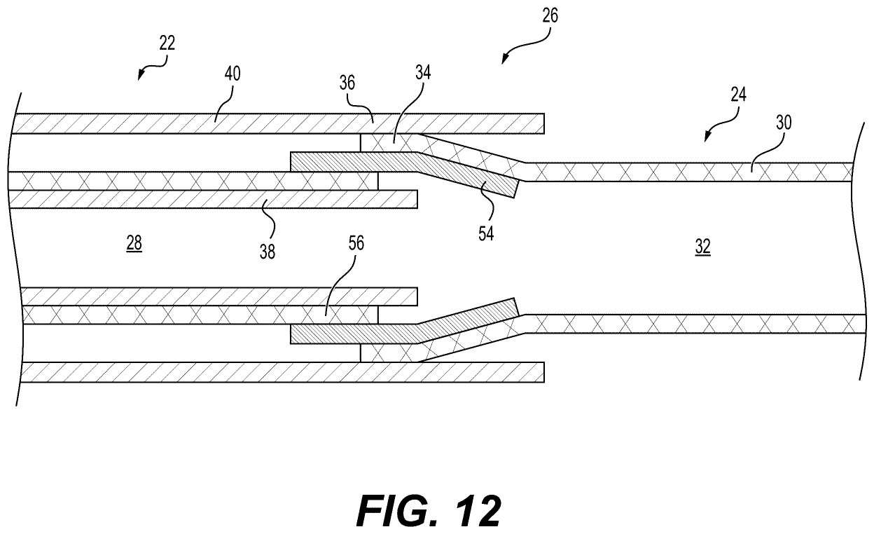

In another implementation, as shown in FIG. 12, a transitionary metal structure 54 can be employed including a metal collar component that can be welded to a metal braid of the expandable layer 30 and also to a reinforcing metal braid 56 for the proximal portion 22. A reflow outer layer 40 may be applied over the assembly.

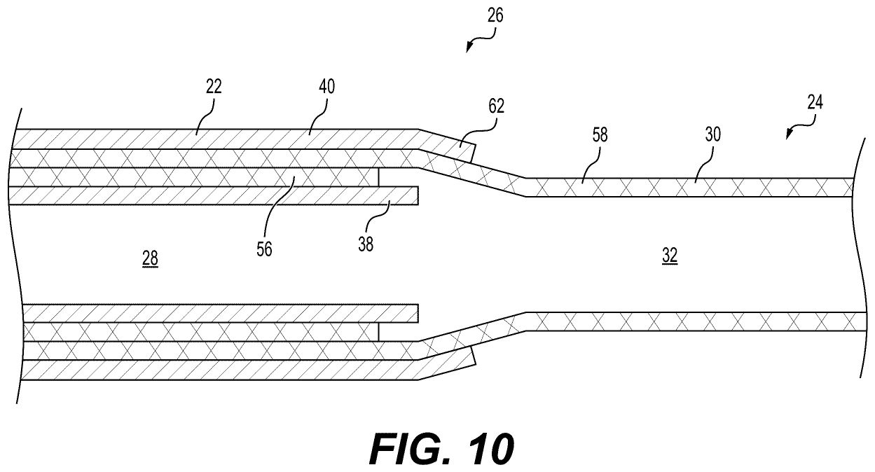

In some implementations, as shown in FIG. 10, the hybrid sheath 8 may include a full-length (such as NiTi) braid 58 that starts at the hub 20 and extends to the distal tip of the hybrid sheath 8. Restated, the expandable layer 30 extends from the expandable distal portion 24 and through the layer structure of the proximal portion 22 all the way to the hub 20. The inner layer 38 and outer layer 40 extend under and over (are overlaid on) the full-length braid. In some implementations, the layers of the proximal portion 22 can be reduced or removed to reduce the profile of the hybrid sheath 8. Use of a full-length braid facilitates easy manufacturing and fixturing in place.

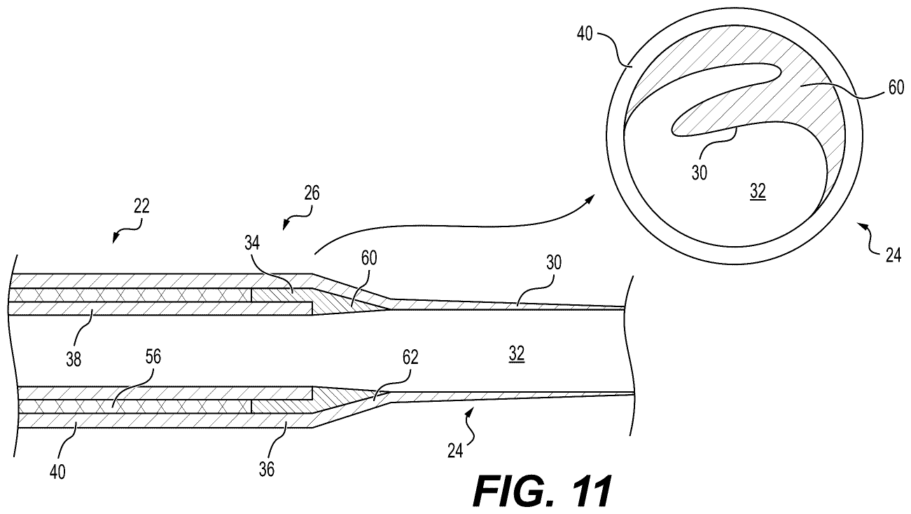

In some implementations, as shown in FIG. 11, the distal portion 24 may be expandable due to unfolding of a normally folded expandable layer 30. The expandable distal portion 24 may include a fold or a crease that opens up during deployment of the implant 12. The proximal end 34 of the expandable distal portion 24 may include a web layer 60 fused to both the proximal end 34 and distal end 36 to form the connector region 26. The web layer is configured to seal the joint even when the distal portion 24 is expanded. For example, TECOFLEX tubing will follow the fold shape when expanding. The length of the seal can be extended by extending the web layer 60 deeper into the proximal portion 22. The web layer 60 can be reflowed into place. In some implementations, the folded section of the distal portion 24 can be terminated with a short rigid section at the proximal end 34. This rigid section can then be overlapped onto the more rigid distal end 36 of the proximal portion 22. Then the layers can be reflowed together.

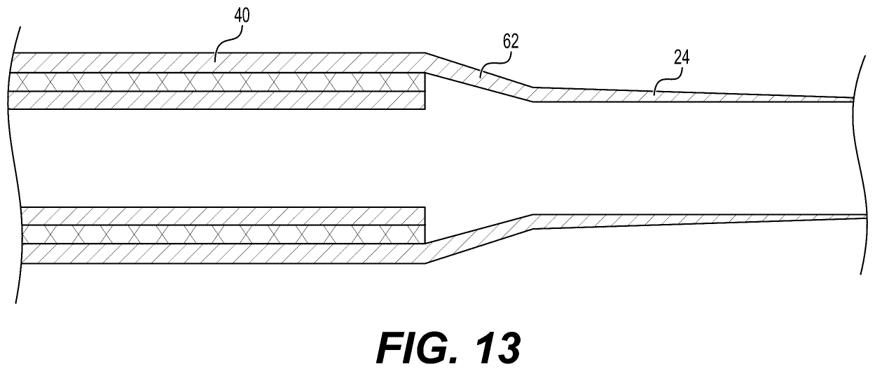

FIG. 13 shows an example implementation wherein the outer layer 40 is a rigid layer reflowed into a tapered transition portion 62 merged with the expandable distal portion 24.

The medical device described herein can include a prosthetic device (for example implant 12) mounted in a radially crimped state on a delivery apparatus (for example, delivery apparatus 10), and the act of advancing the prosthetic device (implant 12) through the lumen of the distal portion 24 comprises advancing the delivery apparatus (for example, delivery apparatus 10) and the prosthetic device through lumen of the distal portion 24 and into the vasculature of the patient. In some examples, the prosthetic device (implant 12) comprises a prosthetic heart valve and the method further comprises implanting the prosthetic heart valve at a treatment site within the patient. As described herein, the prosthetic heart valve is mounted on a balloon catheter of the delivery apparatus (for example, delivery apparatus 10) as the prosthetic heart valve is advanced through the distal portion 24. Also, self-expanding prosthetic heart valves (and other non-balloon implants) could be delivered through the hybrid sheath 8.

### Exemplary Aspects

In view of the described processes and compositions, herein, below are described certain more particularly described aspects of the disclosures. These particularly recited aspects should not, however, be interpreted to have any limiting effect on any different claims containing different or more general teachings described herein, or that the “particular” aspects are somehow limited in some way other than the inherent meanings of the language and formulas literally used therein.

Example 1: A hybrid sheath for deploying a medical device, the hybrid sheath comprising: a proximal portion; an expandable distal portion; and a connector region configured to couple a proximal end of the expandable distal portion to a distal end of the proximal portion to establish communication therethrough.

Example 2: The hybrid sheath according to any example herein, particularly example 1,wherein the proximal portion includes at least one polymer layer defining a first lumen with a fixed diameter; wherein the expandable distal portion defines a second lumen including: an expandable layer, and at least one first polymeric layer combined with the expandable layer; and wherein the connector region couples the proximal end of the expandable distal portion to the distal end of the proximal portion to establish communication between the first and second lumens for passage of the medical device.

Example 3: The hybrid sheath according to any example herein, particularly examples 1-2,wherein the expandable distal portion further includes: an inner liner comprising at least one polymeric layer having an inner surface defining the second lumen; at least one second polymeric layer surrounding the inner liner and wherein the expandable layer surrounds the second polymeric layer; and an outer liner comprising at least one polymeric layer surrounding the first polymeric layer; and wherein the first polymeric layer surrounds the expandable layer; and wherein the inner liner, the second polymeric layer, the expandable layer, the first polymeric layer and the outer liner form a laminate structure configured to expand for passage of the medical device through the second lumen.

Example 4: The hybrid sheath according to any example herein, particularly examples 1-3,further comprising a hub coupled to a proximal end of the proximal portion, the hub configured to engage a stabilizer to resist push forces through the first and second lumens.

Example 5: The hybrid sheath according to any example herein, particularly examples 1-4,wherein the connector region includes an extension of one of the polymeric layers of the expandable distal portion over the distal end of the proximal portion.

Example 6: The hybrid sheath according to any example herein, particularly example 5,wherein the extension of one of the polymeric layers of the expandable distal portion is bonded to the distal end of the proximal portion.

Example 7: The hybrid sheath according to any example herein, particularly example 5,wherein the one of the polymeric layers of the expandable distal portion is the first polymeric layer.

Example 8: The hybrid sheath according to any example herein, particularly example 5,wherein the distal end of the proximal portion extends between the first polymeric layer and the expandable layer at the connector region.

Example 9: The hybrid sheath according to any example herein, particularly example 8,wherein an inner surface of the distal end of the proximal portion is bonded directly to the expandable layer and wherein an inner surface of the first polymeric layer is bonded directly to the distal end of the proximal portion at the connector region.

Example 10: The hybrid sheath according to any example herein, particularly example 9,wherein the first polymeric layer is reflowed onto the distal end of the proximal portion with the expandable layer between the first polymeric layer and the distal end of the proximal portion.

Example 11: The hybrid sheath according to any example herein, particularly examples 1-10,wherein the polymer layer of the proximal portion is a rigid layer.

Example 12: The hybrid sheath according to any example herein, particularly examples 1-11,wherein the expandable layer includes a flexible mesh layer.

Example 13: The hybrid sheath according to any example herein, particularly example 12,wherein the flexible mesh layer includes a braided layer.

Example 14: The hybrid sheath according to any example herein, particularly examples 1-13,wherein the expandable layer includes a folded layer.

Example 15: The hybrid sheath according to any example herein, particularly examples 1-14,wherein the proximal portion includes an inner polymer layer and an outer polymer layer, wherein the inner polymer layer of the proximal portion defines the first lumen and wherein the expandable layer extends between the inner and outer polymer layers of the proximal portion at the connector region.

Example 16: The hybrid sheath according to any example herein, particularly examples 1-15,wherein the first lumen of the proximal portion is 28 French or greater.

Example 17: The hybrid sheath according to any example herein, particularly example 16,wherein a length of the proximal portion is at least 17 inches.

Example 18: The hybrid sheath according to any example herein, particularly examples 1-17,wherein the second lumen of the expandable distal portion has a non-expanded diameter of 20 French or less.

In view of the many possible aspects to which the principles of the disclosed disclosure can be applied, it should be recognized that the illustrated aspects are only preferred examples of the disclosure and should not be taken as limiting the scope of the disclosure. Rather, the scope of the disclosure is defined by the following claims. We, therefore, claim as our disclosure all that comes within the scope and spirit of these claims.

What is claimed is:

1. A hybrid sheath for deploying a medical device, the hybrid sheath comprising:
a proximal portion including at least one polymer layer defining a first lumen with a fixed diameter;
an expandable distal portion defining a second lumen including:
an expandable layer; and
at least one first polymeric layer combined with the expandable layer; and
a connector region configured to couple a proximal end of the expandable distal portion to a distal end of the proximal portion to establish communication between the first and second lumens for passage of the medical device.

2. The hybrid sheath of claim 1, wherein the expandable distal portion further includes:
an inner liner comprising at least one polymeric layer having an inner surface defining the second lumen;
at least one second polymeric layer surrounding the inner liner and wherein the expandable layer surrounds the second polymeric layer; and
an outer liner comprising at least one polymeric layer surrounding the first polymeric layer; and
wherein the first polymeric layer surrounds the expandable layer; and
wherein the inner liner, the second polymeric layer, the expandable layer, the first polymeric layer and the outer liner form a laminate structure configured to expand for passage of the medical device through the second lumen.

3. The hybrid sheath of claim 1, further comprising a hub coupled to a proximal end of the proximal portion, the hub configured to engage a stabilizer to resist push forces through the first and second lumens.

4. The hybrid sheath of claim 1, wherein the connector region includes an extension of one of the polymeric layers of the expandable distal portion over the distal end of the proximal portion.

5. The hybrid sheath of claim 4, wherein the extension of one of the polymeric layers of the expandable distal portion is bonded to the distal end of the proximal portion.

6. The hybrid sheath of claim 4, wherein the one of the polymeric layers of the expandable distal portion is the first polymeric layer.

7. The hybrid sheath of claim 4, wherein the distal end of the proximal portion extends between the first polymeric layer and the expandable layer at the connector region.

8. The hybrid sheath of claim 7, wherein an inner surface of the distal end of the proximal portion is bonded directly to the expandable layer and wherein an inner surface of the first polymeric layer is bonded directly to the distal end of the proximal portion at the connector region.

9. The hybrid sheath of claim 8, wherein the first polymeric layer is reflowed onto the distal end of the proximal portion with the expandable layer between the first polymeric layer and the distal end of the proximal portion.

10. The hybrid sheath of claim 1, wherein the polymer layer of the proximal portion is a rigid layer.

11. The hybrid sheath of claim 1, wherein the expandable layer includes a flexible mesh layer.

12. The hybrid sheath of claim 11, wherein the flexible mesh layer includes a braided layer.

13. The hybrid sheath of claim 1, wherein the expandable layer includes a folded layer.

14. The hybrid sheath of claim 1, wherein the proximal portion includes an inner polymer layer and an outer polymer layer, wherein the inner polymer layer of the proximal portion defines the first lumen and wherein the expandable layer extends between the inner and outer polymer layers of the proximal portion at the connector region.

15. The hybrid sheath of claim 1, wherein the first lumen of the proximal portion is 28 French or greater.

16. The hybrid sheath of claim 15, wherein a length of the proximal portion is at least 17 inches.

17. The hybrid sheath of claim 1, wherein the second lumen of the expandable distal portion has a non-expanded diameter of 20 French or less.
